(19) United States
(12) Patent Application Publication
(10) Pub. No.: US 2011/0034987 A1
(43) Pub. Date: Feb. 10, 2011

(54) ROLL SLEEVE MECHANISM FOR PROXIMAL RELEASE STENT

(76) Inventor: Kenneth C. Kennedy, Clemmons, NC (US)

Correspondence Address: BRINKS HOFER GILSON & LIONE/CHICAGO/COOK, PO BOX 10395, CHICAGO, IL, 60610, US

(21) Appl. No.: 12/846,294

(22) Filed: Jul. 29, 2010

Related U.S. Application Data
(60) Provisional application No. 61/231,142, filed on Aug. 4, 2009.

(51) Int. Cl. A61F 2/84

(52) U.S. Cl. 623/1.11

(57) ABSTRACT

A medical device for releasing expandable prostheses in a proximal to distal direction includes an elongate catheter, a flexible sleeve, and an expandable prosthesis. In one embodiment, the distal end of the flexible sleeve is disposed about the distal end of the elongate catheter. In order to deploy the expandable prosthesis, the flexible sleeve is pulled proximally through the lumen of the elongate catheter to expose the expandable prosthesis. An outer sheath and prosthesis retaining line may also be provided to allow the device to release an expandable prosthesis in the distal to proximal direction, or to allow the recapture of the expandable prosthesis after the flexible sleeve is fully withdrawn into the lumen of the elongate catheter.

### RELATED APPLICATIONS

The present patent document claims the benefit of the filing date under 35 U.S.C. §119(e) of Provisional U.S. Patent Application Ser. No. 61/231,142, filed Aug. 4, 2009, which is hereby incorporated by reference.

### BACKGROUND

The present invention relates generally to medical devices and more particularly to a device for proximally releasing a self-expanding prosthesis.

The use of self-expanding metal stents in body vessels is well-known. Typically, a self-expanding stent is introduced into a body vessel using a push-pull type introducer. In a push-pull device, the self-expanding stent is radially compressed around the outer diameter of an inner catheter and is constrained about the inner catheter by an outer sheath. The introducer is then positioned within a body vessel. Once in position, the stent may be deployed by holding the position of the inner catheter steady with respect to the patient while the outer sheath is withdrawn proximally. As used in the present application, “proximal” and “distal” are defined relative to the operator. As the outer sheath is pulled away from the self-expanding stent, the stent expands radially away from the inner catheter until it is fully uncovered and expanded against the walls of the body vessel.

In a push-pull device, the first portion of the self-expanding stent to make contact with the body vessel is the most distal portion of the stent because the sheath is pulled away from the stent in the proximal direction. This type of system may also be referred to as a “distal release” device. Distal release devices generally allow for accurate placement of the distal portion of a self-expanding stent, but do not allow for accurate placement of the proximal portion of a stent. Accurate placement of the proximal portion of the stent may be important in certain applications.

For example, use of self-expanding stents within the gastrointestinal (GI) tract is well-known. However, the use of a distal release device creates concerns when accurate placement of the proximal portion of the stent is desired. For example, foreshortening stents tend to anchor within the GI tract at the initial location the stent makes contact with the body vessel, and then shorten along their primary axis toward that location. Thus, when using a foreshortening stent, the end that initially opens is relatively easy to place with accuracy, but the final location of the opposite end of the stent is variable.

In some applications there is a clinical need to achieve precise anatomical placement of the proximal end of the self-expanding stent. For example, when there is a need to place a self-expanding stent very high in the esophagus, the proximal end of the stent should ideally be above the stricture but below the cricopharyngeal region of the throat to avoid aggravation of the nerves that control the coughing response. Such need for high esophageal placement of the self-expanding stent often occurs when high strictures develop following surgical esophagectomy. Additionally, high malignant lesions in the esophagus may also be treated with self-expanding stents.

One known method of proximally releasing a self-expanding stent is the Ultraflex™ device from Boston Scientific Corporation. With the Ultraflex™ device the self-expanding stent is radially constrained to the inner catheter of the introducer by knotted string or suture material located within the central region of the self-expanding stent. The string is connected to a user-operable lead string that emerges at the proximal end of the introducer. A series of knots are formed along the introducing catheter such that they may be released one-by-one by pulling the lead string. The first knot to be released is at the proximal edge of the self-expanding stent, with each successive knot being located more distal to its predecessor. The final knot is located near the distal end of the self-expanding stent. As each knot is released, the self-expanding stent begins to expand away from the introducer.

There are several disadvantages associated with the Ultraflex release method. For example, string or suture material may become entangled, break during deployment, or fail to release. Deployment of the stent may also be choppy and irregular because each knot is released individually. Additionally, the stent cannot be recaptured after release of the knots of the Ultraflex device. Thus, there is a need for a proximal release device which reduces the risk of malfunction, provides for a smoother deployment, and allows for the recapture of the stent after partial deployment.

### BRIEF SUMMARY

The invention may include any of the following aspects in various combinations and may also include any other aspect described below in the written description or in the attached drawings.

A medical device includes an elongate catheter, an expandable prosthesis disposed about the distal portion of the elongate catheter, and a flexible sleeve everted upon itself. The medical device has a contracted delivery configuration and an expanded delivery configuration. The flexible sleeve has a first and second portion and a distal end. The distal end of the flexible sleeve is disposed about the distal end of the elongate catheter. The first portion of the flexible sleeve is disposed about the expandable prosthesis in the contracted delivery configuration; the second portion of the flexible sleeve is disposed about the first portion of the flexible sleeve in the contracted delivery configuration, and a portion of the second portion of the flexible sleeve is disposed within the lumen of the elongate catheter. The flexible sleeve is configured to be pulled proximally within the lumen of the elongate catheter such that at least a portion of the second portion of the flexible sleeve is disposed within the elongate catheter in the expanded delivery configuration.

The flexible sleeve may be composed of expanded polytetrafluorethylene.

The medical device may further include an outer sheath disposed about the elongate catheter and flexible sleeve.

The medical device may also include an atraumatic tip disposed on the distal end of the elongate catheter such that the atraumatic tip forms a generally smooth transition between the atraumatic tip and outer sheath.

The medical device may also include a prosthesis retaining line attached to the expandable prosthesis and configured to release the expandable prosthesis.

The medical device may also include a handle which is in communication with the second portion of the flexible sleeve, such that when the handle is actuated the second portion of the flexible sleeve rolls in the distal direction and is pulled proximally into the elongate catheter.

Another embodiment of the medical device includes an elongate catheter, an expandable prosthesis disposed about the distal portion of the elongate catheter, and a flexible sleeve everted about the distal end of the elongate catheter. The prosthesis delivery system has a contracted delivery configuration and an expanded delivery configuration. The flexible sleeve has a proximal and a distal portion. The distal portion of the flexible sleeve is disposed about the expandable prosthesis and the proximal portion of the flexible sleeve is disposed within the lumen of the elongate catheter in the contracted delivery configuration. The flexible sleeve is configured to be pulled within the lumen of the elongate catheter such that at least a portion of the distal portion of the flexible sleeve is disposed within the lumen of the catheter in the expanded delivery configuration.

This embodiment of the medical device may include a flexible sleeve composed of expanded polytetrafluroethylene, an outer sheath disposed about the elongate catheter and flexible sleeve, an atraumatic tip at the distal end of the elongate catheter, and a prosthesis retaining line attached to the expandable prosthesis.

This embodiment of the medical device may also include a handle which is in communication with the proximal portion of the flexible sleeve such that when the handle is actuated the distal portion of the flexible sleeve is pulled proximally into the elongate catheter.

### BRIEF DESCRIPTION OF THE DRAWINGS

FIG. 1 is a broken longitudinal section view of the medical device.

FIG. 2 is a partial longitudinal section view of the proximal release introducer wherein the expandable prosthesis is partially deployed.

FIG. 3 is a partial perspective view of the rotational handle assembly.

FIG. 4 is a partial longitudinal section view of the proximal release introducer including an outer sheath.

FIG. 5 is a partial longitudinal section view of the proximal release introducer wherein the elongate catheter includes an atraumatic tip.

FIG. 6 is a partial longitudinal section view of the proximal release introducer including a prosthesis retaining line.

FIG. 7 is a partial longitudinal section view of the proximal release introducer including an outer sheath and prosthesis retaining line wherein the expandable prosthesis is partially deployed.

FIG. 8 is a partial longitudinal section view of the proximal release introducer including an outer sheath and a prosthesis retaining line wherein the expandable prosthesis is partially recaptured.

FIG. 9 is a partial longitudinal section view of the proximal release introducer including an outer sheath and a prosthesis retaining line wherein the prosthesis is fully recaptured.

FIG. 10 is a broken longitudinal section view of the medical device wherein the flexible sleeve is everted about the distal end of the elongate catheter.

FIG. 11 is a longitudinal section view of the proximal release introducer wherein the flexible sleeve is everted about the distal end of the elongate catheter and the proximal release introducer includes an atraumatic tip, an outer sheath, and a prosthesis retaining line.

### DETAILED DESCRIPTION OF THE DRAWINGS AND THE PRESENTLY PREFERRED EMBODIMENTS

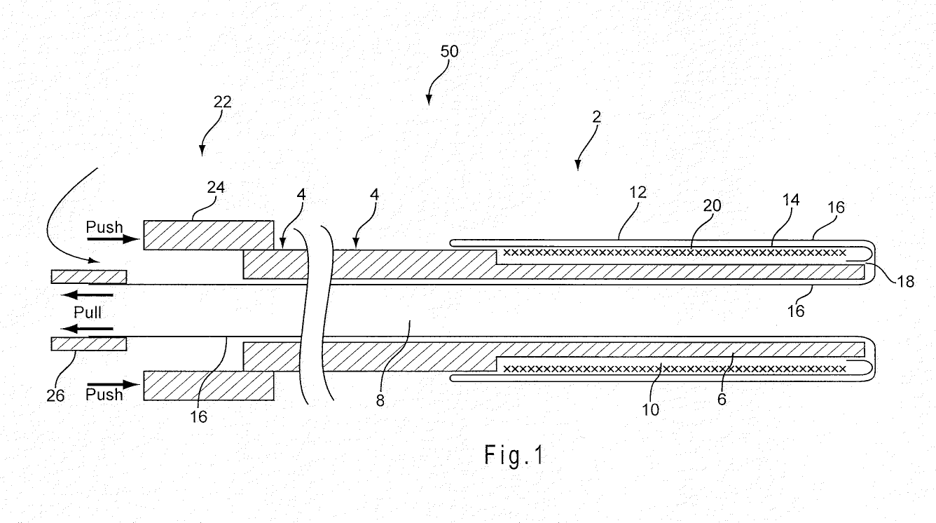

Referring now to the figures, a medical device with a proximal release introducer is shown. One embodiment of the medical device is shown in FIG. 1. Medical device 50 includes a proximal release introducer 2. Further embodiments of medical device 50 may also include a handle assembly such as handle assembly 22 or rotational handle assembly 28. Proximal release introducer 2 includes an elongate catheter 4 with a prosthesis receiving region 10 disposed upon the distal end 6 of elongate catheter 4 and an inner lumen 8. An expandable prosthesis 20, radially compressed to a diameter near to that of elongate catheter 4, is disposed within prosthesis receiving region 10. Expandable prosthesis 20 is any device placed within a body vessel to replace or supplement the function of the body vessel, including stents. Disposed over expandable prosthesis 20, and thereby constraining expandable prosthesis 20 to prosthesis receiving region 10, is flexible sleeve 12. Flexible sleeve 12 is a thin-walled tube of flexible material everted upon itself. The distal end 18 of flexible sleeve 12 is disposed about distal end 6 of elongate catheter 4. Distal end 18 of flexible sleeve 12 may be affixed to distal end 6 of elongate catheter 4 by any of a number of well-known methods, including the use of adhesives and sutures. Distal end 18 of flexible sleeve 12 may also be disposed between and in contact with distal end 6 of elongate catheter 4 and expandable prosthesis 20 and held in place by frictional forces. Flexible sleeve 12 may be made of any suitable flexible material, including expanded polytetrafluorethylene (ePTFE). The wall thickness of flexible sleeve 12 depends on the material used. For ePTFE, a typical wall thickness would be between .001″-.01″, although some applications may require greater or lesser thickness.

Flexible sleeve 12 has a first portion 14 and a second portion 16. First portion 14 of flexible sleeve 12 is disposed adjacent to expandable prosthesis 20. Second portion 16 of flexible sleeve 12 includes the portion of flexible sleeve 12 everted upon the first portion 14 as well a portion of flexible sleeve 12 which is disposed within the inner lumen 8 of elongate catheter 4.

Medical device 50 may include proximal release introducer 2 connected to a handle assembly 22. Handle assembly 22 includes a pusher portion 24 and a puller portion 26. Pusher portion 24 is attached to elongate catheter 4. Puller portion 26 is in communication with second portion 16 of flexible sleeve 12. In operation, after placing proximal release introducer 2 into a patient, the pusher portion 24 is held in position while the puller portion 26 is withdrawn proximally. As puller portion 26 is withdrawn proximally, flexible sleeve 12 is also withdrawn proximally through the inner lumen 8 of elongate catheter 4.

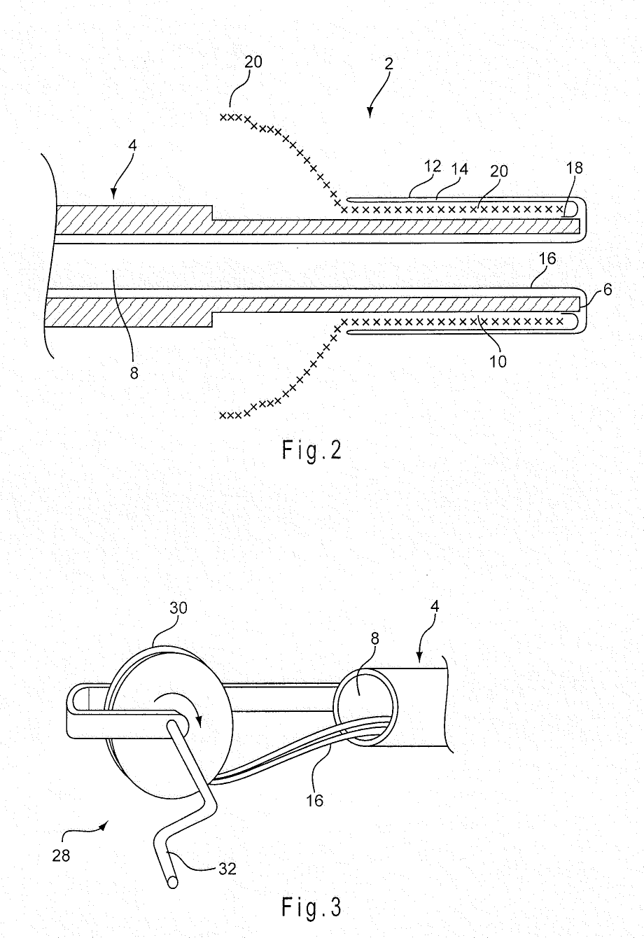

In FIG. 2, the handle assembly 22 (not shown) of the medical device 50 has been partially actuated so that the second portion 16 of flexible sleeve 12 has been partially withdrawn into lumen 8 of elongate catheter 4. As this occurs, the flexible sleeve 12 rolls upon itself releasing expandable prosthesis 20. The use of lubricious material for the flexible sleeve 12 may encourage the second portion 16 of flexible sleeve 12 to slide upon the first portion 14 of flexible sleeve 12, but the use of lubricious material is not required for flexible sleeve 12 to roll upon itself. As the flexible sleeve 12 rolls upon itself, second portion 16 of flexible sleeve 12 is pulled into the inner lumen 8 of elongate catheter 4, releasing expandable prosthesis 20 in the proximal to distal direction. Nearly all of flexible sleeve 12 may eventually be pulled proximally into the inner lumen 8 of elongate catheter 4.

An alternate embodiment of a handle assembly is shown in FIG. 3. Rotational handle assembly 28 includes a take-up spool 30 and a crank handle 32. Second portion 16 of flexible sleeve 12 is attached to take-up spool 30 so that when crank handle 32 is operated to rotate take up spool 30, the second portion 16 of flexible sleeve 12 will be proximally withdrawn through lumen 8 of elongate catheter 4 and taken up by take-up spool 30. Those skilled in the art will appreciate that rotation take-up mechanisms such as rotational handle assembly 28 may be easily configured into other types of handle constructs and may be actuated in other ways. For example, a handle utilizing a pistol style grip, such as the device in U.S. application Ser. No. 11/879,176 to Keady et al. may be used. As shown in Keady, the handle utilizing a pistol grip may be further configured with a trigger mechanism that converts trigger squeezes into rotary motion within the handle. A pistol-style grip may also be configured to convert trigger squeezes into linear motion so as to operate with a push-pull handle assembly such as handle assembly 22.

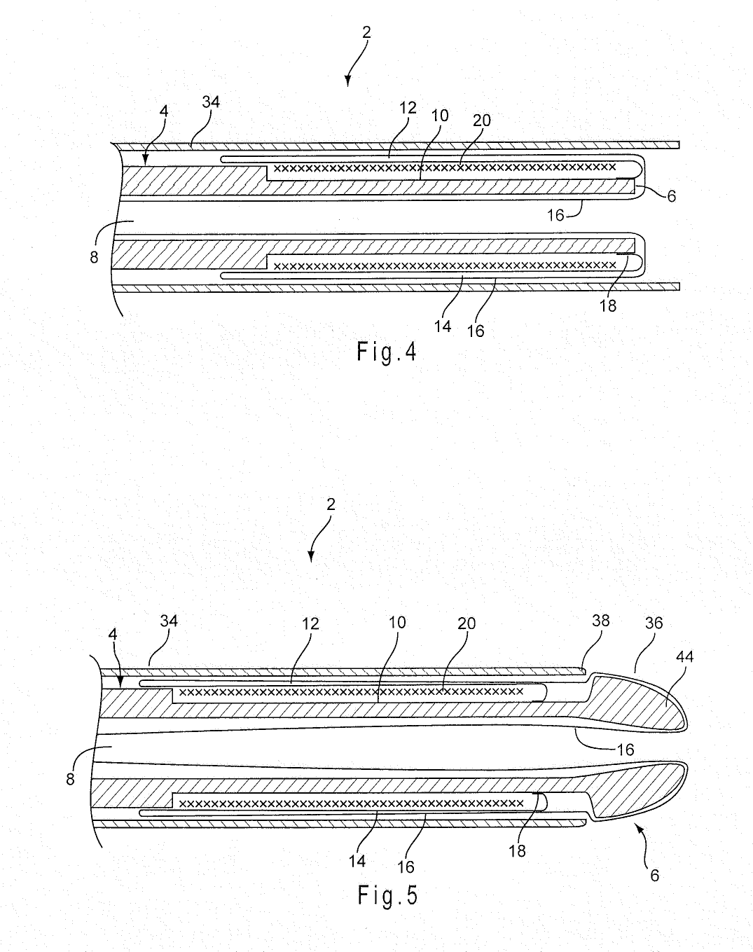

FIG. 4 illustrates another embodiment of proximal release introducer 2 wherein a proximally retractable outer sheath 34 is disposed over the proximal release introducer 2 shown in FIG. 1. Outer sheath 34 is disposed over flexible sleeve 12 to aid in the constraint of expandable prosthesis 20. This may prevent expandable prosthesis 20 from detrimentally stretching or embedding into flexible sleeve 12.

FIG. 5 illustrates another embodiment of proximal release introducer 2 wherein the portion 36 of elongate catheter 4 distal of the prosthesis receiving region 10 is formed into an atraumatic tip 44. An outer sheath 34 is disposed over flexible sleeve 12. The atraumatic tip 44 extends distally beyond the distal end 38 of outer sheath 34. When outer sheath 34 is fully extended to its most distal position, the shape of atraumatic tip 44 forms a generally smooth transition with the distal end 38 of outer sheath 34. Because flexible sleeve 12 is formed from a thin, flexible material, it may easily conform to the tortuous path around atraumatic tip 44. Atraumatic tip 44 may be formed by any well-known forming technique, including machining and thermoplastic molding. Additionally, atraumatic tip 44 could be integrally formed as part of elongate catheter 4 or formed separately from elongate catheter 4. If atraumatic tip 44 is formed separately from elongate catheter 4 it may be advantageous to manufacture atraumatic tip 44 from a different material than elongate catheter 4. Examples of potentially advantageous materials for atraumatic tip 44 include lubricious materials, polyethylene, and PTFE.

The embodiments of proximal release introducer 2 incorporating an outer sheath 34 may also be used as a distal release introducer. When an operator desires to release an expandable prosthesis 20 in the proximal to distal direction, the outer sheath 34 is first withdrawn proximally so that outer sheath 34 is no longer constraining expandable prosthesis 20. Expandable prosthesis 20 may then be released by proximally withdrawing second portion 16 of flexible sleeve 12 through the lumen 8 of elongate catheter 4. However, when an operator desires to release an expandable prosthesis 20 in the distal to proximal direction, the flexible sleeve 12 is first withdrawn proximally through the lumen 8 of elongate catheter 4 with outer sheath 34 disposed about expandable prosthesis 20. With the flexible sleeve 12 completely withdrawn, the proximal release introducer 2 may release expandable prosthesis 20 in a typical distal to proximal direction by proximally withdrawing outer sheath 34.

As shown in FIG. 7, the embodiments of proximal release introducer 2 incorporating an outer sheath 34 may also be used to release an expandable prosthesis 20 from the middle of the expandable prosthesis 20. When an operator desires to release an expandable prosthesis 20 from the middle, outer sheath 34 is withdrawn in the proximal direction so that it constrains only a proximal portion of expandable prosthesis 20. The operator also proximally withdraws second portion 16 of flexible sleeve 12 through the lumen 8 of elongate catheter 4 such that flexible sleeve 12 constrains only a distal portion of expandable prosthesis 20. By constraining the proximal and distal portions, but not the middle portion, of expandable prosthesis 20, the operator allows the middle portion of expandable prosthesis 20 to be released first. The operator may then further withdraw outer sheath 34 and flexible sleeve 12 to fully release expandable prosthesis 20, or the operator may push outer sheath 34 in the distal direction to recapture expandable prosthesis 20. Recapture of expandable prosthesis 20 may be assisted by the use of a prosthesis retaining line 40, as discussed below.

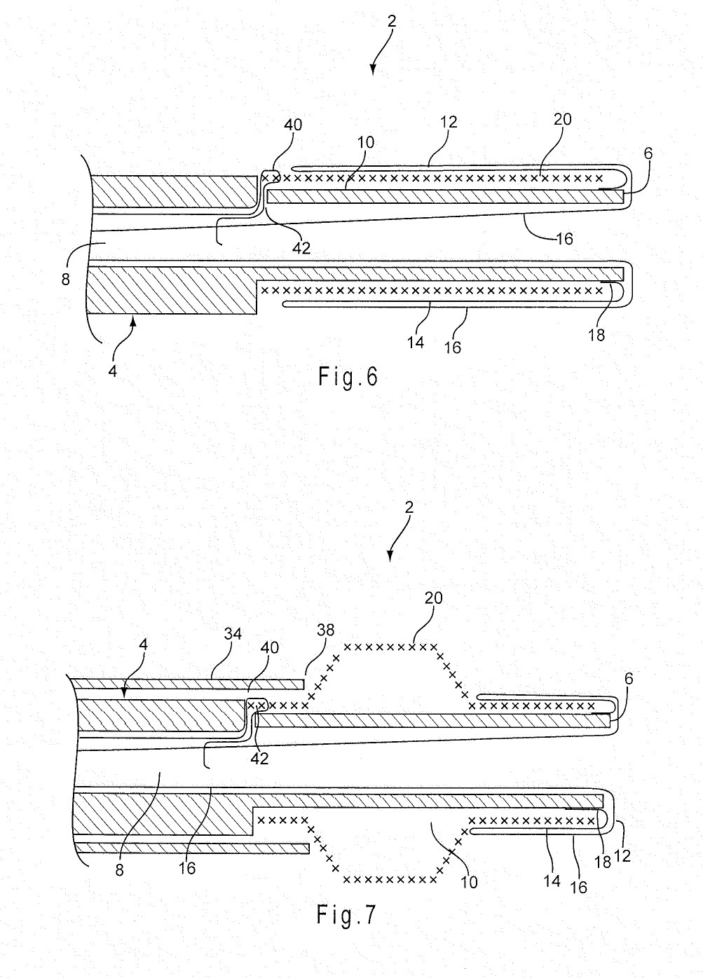

FIG. 6 illustrates another embodiment of medical device 50 which includes a prosthesis retaining line 40. Prosthesis retaining line 40 traverses from a position near the handle assembly 22 (not shown) through the lumen 8 of elongate catheter 4 to the proximal release introducer 2. Prosthesis retaining line 40 passes through an opening 42 that forms a communication between lumen 8 of elongate catheter 4 and prosthesis receiving region 10. Prosthesis retaining line 40 is formed into a loop which captures the expandable prosthesis 20. During operation of the proximal release introducer 2, prosthesis retaining line 40 retains the expandable prosthesis 20 even after expandable prosthesis 20 has been allowed to radially expand away from prosthesis receiving region 10 of elongate catheter 4. If a complete release of the expandable prosthesis 20 is desired, the operator may pull on the proximal end of prosthesis retaining line 40 to disengage the prosthesis retaining line 40 from expandable prosthesis 20. Prosthesis retaining line 40 may be made of fine metallic wire, cable, suture, or string, among other materials.

FIG. 7 illustrates another embodiment of proximal release introducer 2 which includes a prosthesis retaining line 40 and an outer sheath 34. The combination of prosthesis retaining line 40 and outer sheath 34 enables the proximal release introducer 2 to recapture expandable prosthesis 20 after partial or complete withdrawal of flexible sleeve 12 through lumen 8 of elongate catheter 4. In FIG. 7, second portion 16 of flexible sleeve 12 has been partially withdrawn into lumen 8 of elongate catheter 4 and therefore has partially released expandable prosthesis 20. Prosthesis retaining line 40 remains attached to expandable prosthesis 20. As outer sheath 34 is pushed distally, it recaptures expandable prosthesis 20.

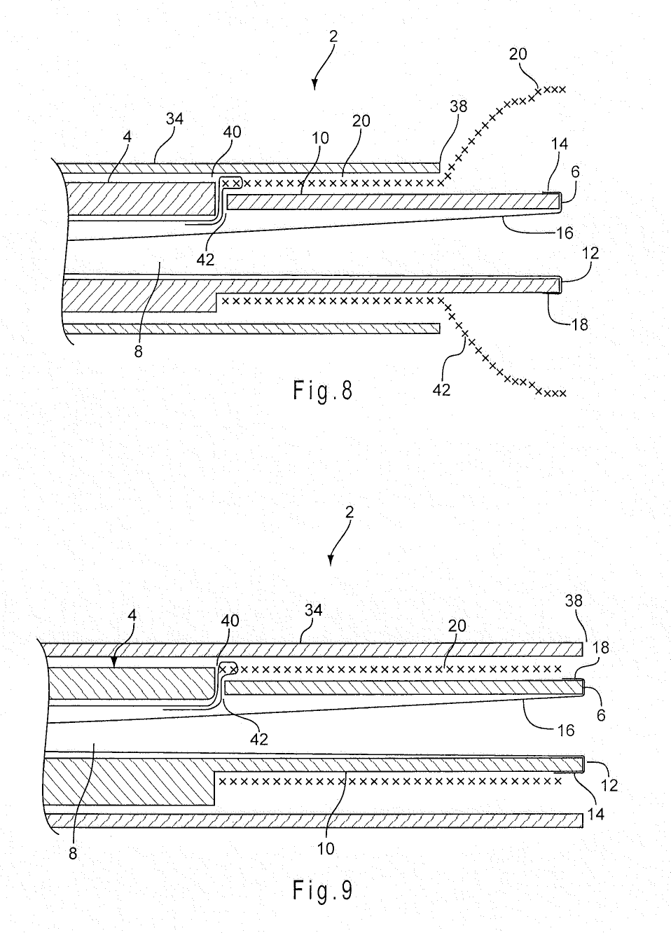

In FIG. 8, flexible sleeve 12 has been fully withdrawn into lumen 8 of elongate catheter 4. Outer sheath 34 recaptures expandable prosthesis 20 as it is pushed in the distal direction. In FIG. 9, expandable prosthesis 20 has been completely recaptured by outer sheath 34. Once expandable prosthesis 20 is fully recaptured proximal release introducer 2 may be repositioned within the patient. Once proximal release introducer 2 is properly positioned, outer sheath 34 may be withdrawn proximally to release expandable prosthesis 20 in the distal to proximal direction. However, once prosthesis retaining line 40 is disengaged from expandable prosthesis 20, the ability to recapture the expandable prosthesis may be compromised or lost.

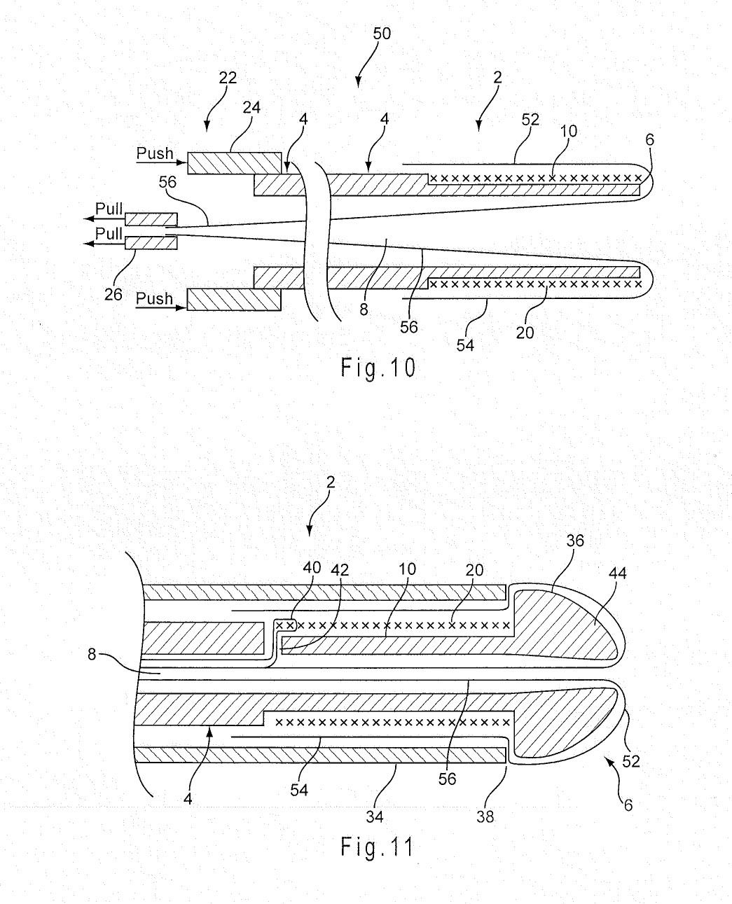

Yet another embodiment of medical device 50 is shown in FIG. 10. Proximal release introducer 2 includes an elongate catheter 4 with a prosthesis receiving region 10 disposed upon the distal end 6 of elongate catheter 4, and an inner lumen 8. An expandable prosthesis 20, radially compressed to a diameter near to that of elongate catheter 4, is disposed within prosthesis receiving region 10. Expandable prosthesis 20 includes any device placed within a body vessel to replace or supplement the function of the body vessel, including stents. Disposed over expandable prosthesis 20, and thereby constraining expandable prosthesis 20 to prosthesis receiving region 10, is flexible sleeve 52. Flexible sleeve 52 is a thin-walled tube of flexible material with a distal portion 54 and proximal portion 56. Flexible sleeve 52 is everted about the distal end 6 of elongate catheter 4 such that distal portion 54 of flexible sleeve 52 is disposed about expandable prosthesis 20 and proximal portion 56 of flexible sleeve 52 is disposed within the inner lumen 8 of elongate catheter 4. Flexible sleeve 52 may be made of any flexible material, including expanded polytetrafluorethylene (ePTFE).

Medical device 50 may include proximal release introducer 2 connected to a handle assembly 22. Handle assembly 22 includes a pusher portion 24 and a puller portion 26. Pusher portion 24 is attached to elongate catheter 4. Puller portion 26 is in communication with proximal portion 56 of flexible sleeve 52. In operation, after placing proximal release introducer 2 into a patient, the pusher portion 24 is held in position while the puller portion 26 is withdrawn proximally. As puller portion 26 is withdrawn proximally, flexible sleeve 52 is also withdrawn proximally through the inner lumen 8 of elongate catheter 4. As flexible sleeve 52 is withdrawn proximally through inner lumen 8 of elongate catheter 4, the distal portion 54 of flexible sleeve 52 is withdrawn into inner lumen 8 of elongate catheter 4, releasing expandable prosthesis 20 in the proximal to distal direction.

Further embodiments of a proximal release introducer with a flexible sleeve 52 are shown in FIG. 11. Such a proximal release introducer may incorporate any or all of the following, as described in previous paragraphs: an outer sheath 34, a prosthesis retaining line 40, and an atraumatic tip 44. An embodiment of proximal release introducer 2 with a flexible sleeve 52 that incorporates an outer sheath 34 may release an expandable prosthesis 20 in the proximal to distal or distal to proximal directions, An embodiment of proximal release introducer 2 with a flexible sleeve 52 that incorporates an outer sheath 34 may also release an expandable prosthesis 20 from the middle of the expandable prosthesis 20 as described in previous paragraphs.

While preferred embodiments of the invention have been described, it should be understood that the invention is not so limited, and modifications may be made without departing from the invention. The scope of the invention is defined by the appended claims, and all devices that come within the meaning of the claims, either literally or by equivalence, are intended to be embraced therein. Furthermore, the advantages described above are not necessarily the only advantages of the invention, and it is not necessarily expected that all of the described advantages will be achieved with every embodiment of the invention.

1. A medical device comprising:
an elongate catheter having a distal end and a lumen extending therethrough;
a prosthesis receiving region disposed on said distal end of said elongate catheter;
an expandable prosthesis disposed about said prosthesis receiving region of said elongate catheter;
said medical device having a contracted delivery configuration and expanded delivery configuration;
a flexible sleeve everted upon itself, said flexible sleeve having a first and second portion and a distal end, said distal end of said flexible sleeve disposed about said distal end of said elongate catheter;
said first portion of said flexible sleeve being disposed about said expandable prosthesis in said contracted delivery configuration;
said second portion of said flexible sleeve being disposed about said first portion of said flexible sleeve in said contracted delivery configuration, wherein said second portion of said flexible, sleeve may be partially disposed within said lumen of said elongate catheter; and
said flexible sleeve configured to be pulled proximally within said lumen of said elongate catheter such that at least a portion of said second portion of said flexible sleeve is disposed within said elongate catheter in the expanded delivery configuration.

2. The medical device of claim 1 wherein said flexible sleeve is composed of expanded polytetrafluorethylene.

3. The medical device of claim 1 further comprising an outer sheath disposed about said elongate catheter and said flexible sleeve in the contracted delivery configuration.

4. The medical device of claim 3 wherein at least a portion of said elongate catheter distal of said prosthesis receiving region has substantially the same diameter as said outer sheath.

5. The medical device of claim 1 further comprising a prosthesis retaining line, said prosthesis retaining line attached to said expandable prosthesis and configured to release said expandable prosthesis.

6. The medical device of claim 1 further comprising a handle, said handle in communication with said second portion of said flexible sleeve such that when said handle is actuated said second portion of said flexible sleeve is pulled proximally within said elongate catheter.

7. A medical device comprising:
an elongate catheter having a distal end and a lumen;
an expandable prosthesis, said expandable prosthesis disposed upon said distal end of said elongate catheter;
a flexible sleeve everted upon itself, said flexible sleeve having a first portion and a second portion;
said first portion of said flexible sleeve disposed about said expandable prosthesis in a contracted delivery configuration;
said second portion of said flexible sleeve configured to be slidably urged within said lumen of said elongate catheter such that at least a portion of said second portion of said flexible sleeve is disposed within said elongate catheter in an expanded delivery configuration.

8. The medical device of claim 7 wherein said flexible sleeve is composed of expanded polytetrafluorethylene.

9. The medical device of claim 7 further comprising an outer sheath disposed about said elongate catheter and said flexible sleeve.

10. The medical device of claim 9 wherein at least a portion of said elongate catheter distal of said expandable prosthesis has substantially the same diameter as said outer sheath.

11. The medical device of claim 7 further comprising a prosthesis retaining line, said prosthesis retaining line attached to said expandable prosthesis and configured to release said expandable prosthesis.

12. The medical device of claim 7 further comprising a handle, said handle in communication with said second portion of said flexible sleeve such that when said handle is actuated said second portion of said flexible sleeve is pulled proximally within said elongate catheter.

13. A medical device comprising:
an elongate catheter having a distal end and a lumen extending therethrough;
a prosthesis receiving region disposed on said distal end of said elongate catheter;
an expandable prosthesis disposed about said prosthesis receiving region of said elongate catheter;
said medical device having a contracted delivery configuration and an expanded delivery configuration;
a flexible sleeve having a distal portion and a proximal portion, said flexible sleeve everted about said distal end of said elongate catheter in said contracted delivery configuration such that said distal portion of said flexible sleeve is disposed about said expandable prosthesis and said proximal portion is disposed within said lumen of said elongate catheter;
said flexible sleeve configured to be pulled proximally within said lumen of said elongate catheter such that at least a portion of said distal portion of said flexible sleeve is disposed within said elongate catheter in the expanded delivery configuration.

14. The medical device of claim 13 wherein said flexible sleeve is composed of expanded polytetrafluorethylene.

15. The medical device of claim 13 further comprising an outer sheath disposed about said elongate catheter and said flexible sleeve in the contracted delivery configuration.

16. The medical device of claim 15 wherein at least a portion of said elongate catheter distal of said prosthesis receiving region has substantially the same diameter as said outer sheath.

17. The medical device of claim 13 further comprising a prosthesis retaining line, said prosthesis retaining line attached to said expandable prosthesis and configured to release said expandable prosthesis.

18. The medical device of claim 13 further comprising a handle, said handle in communication with said proximal portion of said flexible sleeve such that when said handle is actuated said proximal portion of said flexible sleeve is pulled proximally within said elongate catheter.
